(19) United States
(12) Patent Application Publication
(10) Pub. No.: US 2017/0119270 A1
(43) Pub. Date: May 4, 2017

(54) SYSTEM AND METHOD FOR ELECTRIC BRAIN STIMULATOR

(71) Applicant: National Central University, Taoyuan City (TW)

(72) Inventors: Chi-Hung JUAN, Taoyuan City (TW); Norden E. HUANG, Taoyuan City (TW); Wei-Kuang LIANG, Taoyuan City (TW)

(21) Appl. No.: 14/994,517

(22) Filed: Jan. 13, 2016

(30) Foreign Application Priority Data
Oct. 30, 2015 (TW) 104135832

(51) Int. Cl. A61B 5/04; A61B 5/0478; A61B 5/0484

(52) U.S. Cl. CPC A61B 5/04012; A61B 5/0484; A61B 5/0478

(57) ABSTRACT

The invention provides a method for electric brain stimulator. In the beginning, obtaining a brain functional amplitude modulation spectrum, wherein the brain functional amplitude modulation spectrum is a relationship between carrier frequency and amplitude-frequency on different brain sites. Then selecting a first alternating current frequency, wherein the first alternating current frequency is determined by the amplitude-frequency in which the brain functional amplitude modulation spectrum display a maximum power relation value, or a maximum correlation value with any behavior index of behavior and cognitive functions. And selecting a second alternating current frequency, wherein the second alternating current frequency is determined by the carrier frequency in which the brain functional amplitude modulation spectrum display a maximum power relation value, or a maximum correlation value with any behavior index of behavior and cognitive functions. In the end, outputting an alternating current signal, the alternating current signal is generated based on a first cosine function of the first alternating current frequency, a second cosine function of the second alternating current frequency, or a combination of the first cosine function and the second cosine function.

### CROSS-REFERENCE TO RELATED APPLICATIONS

This Non-provisional application claims priority under 35 U.S.C. §119(a) on Patent Application No(s). [104135832] filed in Taiwan, Republic of China [Oct. 30, 2015], the entire contents of which are hereby incorporated by reference.

### FIELD OF THE INVENTION

The invention relates to a system and a method for electric brain stimulator. In particular, to output an alternating current signal based on a brain functional amplitude modulation spectrum.

### BACKGROUND OF THE INVENTION

Brain stimulation techniques such as transcranial direct current stimulation (tDCS), transcranial alternating current stimulation (tACS), transcranial magnetic stimulation (TMS), and ultrasonic neuromodulation (UNMOD) are useful tools to alter neural activities in the brain, and thereby alter/improve cognitive performance and behaviors.

Stimulation current or pulses can be applied either continuously or rhythmically in order to achieve continuous activation/deactivation through neural entrainment in the targeted brain region. For example, multiple TMS pulses delivered every 100 millisecond (10 Hz) can induce action potentials of the same rate. Similarly, tACS current in the form of 10 Hz cycle can also induce changes in neural activity that corresponds to alpha band (roughly 10 Hz) brain wave signals when measured via electroencephalogram or magnetoencephalogram (EEG/MEG). For lack of detailed understanding of the dynamic brain functions, there is no scientific base to select the stimulating signals in order to achieve the desired outcomes: all the above mentioned techniques are operated on a pure try-and-error, hit-and-miss based.

Please refer FIG. 1A, 1B and 1C, FIG. 1A and 1B illustrates a Holo-Hilbert Spectrum in the prior art. FIG. 1C illustrates the example K-value corresponding to the Holo-Hilbert Spectrum of FIG. 1A and 1B. FIG. 1A illustrates a Holo-Hilbert Spectrum 100 that utilizes anodal transcranial direct current stimulation (a-tDCS) and sham transcranial direct current stimulation for a person suffering from poor memory (low performers) in the prior art. FIG. 1B illustrates a Holo-Hilbert Spectrum 110 that utilizes anodal transcranial direct current stimulation (a-tDCS) and sham transcranial direct current stimulation for a person with good memory (high performers) in the prior art. With reference to FIG. 1C, the difference of K-values 120, 122 for low performers between anodal transcranial direct current stimulation (a-tDCS) and sham transcranial direct current stimulation is 0.002 (P=0.002). As result, the memory is improved for the person suffering from poor memory. With further reference to FIG. 1C, the K-values 130, 132 between anodal transcranial direct current stimulation (a-tDCS) and sham transcranial direct current stimulation does not provide any memory improvement for high performers. Therefore, previous tDCS has shown to improve memory for low performers sometimes, but had no, or even slightly degrading, effect on the high performers.

### SUMMARY OF THE INVENTION

The present invention provides a method for electric brain stimulator in a brain stimulator device, comprises obtaining a brain functional amplitude modulation spectrum, wherein the brain functional amplitude modulation spectrum comprises a power relation value or a correlation value of behavior and cognitive function between a frequency range and an amplitude-frequency range on different brain sites.

Then determining a first alternating current frequency, wherein the first alternating current frequency is determined by the amplitude-frequency range corresponding to a maximum power relation value or a maximum correlation value of behavior and cognitive function in the brain functional amplitude modulation spectrum.

The method further comprises determining a second alternating current frequency, wherein the second alternating current frequency is determined by the frequency range corresponding to a maximum power relation value or the maximum correlation value of behavior and cognitive function in the brain functional amplitude modulation spectrum.

And outputting an alternating current signal, wherein the alternating current signal is generated based on a first cosine function of the first alternating current frequency, a second cosine function of the second alternating current frequency, or a combination of the first cosine function and the second cosine function.

In an embodiment of the invention, a system of electric brain stimulator comprises a detection unit, an analysis unit, a selection unit and an electronic shock unit.

The detection unit acquires a plurality of brainwave data.

The analysis unit is connected to the detection unit for analyzing the plurality of brainwave data to obtain a brain functional amplitude modulation spectrum. The brain functional amplitude modulation spectrum comprises a power relation value or a correlation value of behavior and cognitive function between a frequency range and an amplitude-frequency range on different brain sites. The analysis unit also outputs an alternating current signal, wherein the alternating current signal is generated based on a first cosine function of the first alternating current frequency, a second cosine function of the second alternating current frequency, or a combination of the first cosine function and the second cosine function.

The selection unit is connected to the analysis unit for determining a first alternating current frequency based on the amplitude-frequency range corresponding to a maximum power relation value or a maximum correlation value of behavior and cognitive function in the brain functional amplitude modulation spectrum. The selection unit determines a second alternating current frequency based on the frequency range corresponding to a maximum power relation value or a maximum correlation value of behavior and cognitive function in the brain functional amplitude modulation spectrum.

The electronic shock unit is connected to the analysis unit for outputting an electrical current directly applied the scalp, wherein the electrical current corresponding to the alternating current signal.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. The patent or application file contains at least one drawing executed in color. Copies of this patent or patent application publication with color drawing(s) will be provided by the Office upon request and payment of the necessary fee.

FIG. 1A and 1B illustrates the example of Holo-Hilbert Spectrum.

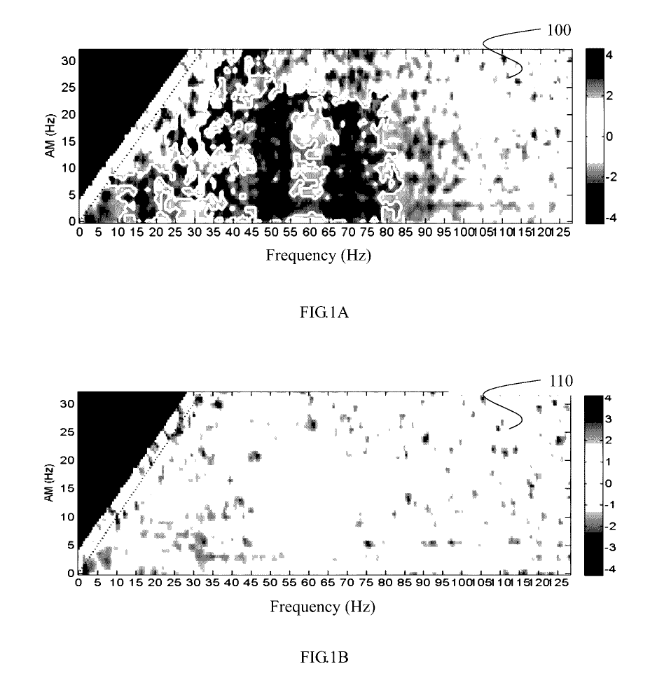

FIG. 1C illustrates the example K-value corresponding to the Holo-Hilbert Spectrum of FIG 1A and 1B.

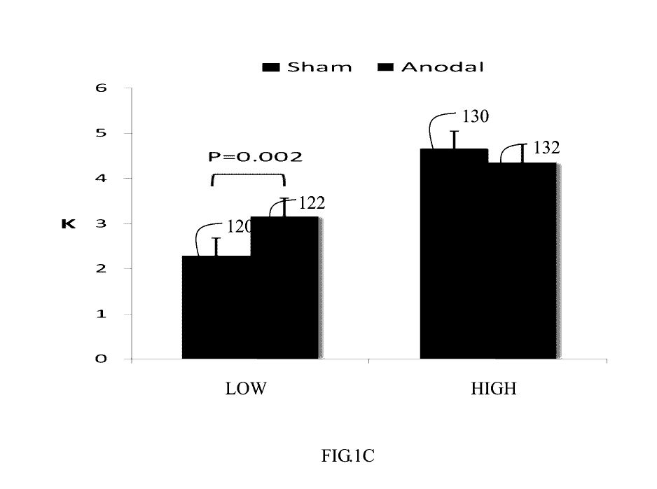

FIG. 2 is a block diagram of a system in which embodiments of electric brain stimulator in accordance with various embodiments of the present disclosure.

FIG. 3 illustrates the example the Binding Visual Working Memory Paradigm in accordance with various embodiments of the present disclosure.

FIG. 4 illustrates a brain functional amplitude modulation spectrum 410 with correlation between power and K-value in accordance with various embodiments of the present disclosure.

FIG. 5 illustrates another brain functional amplitude modulation spectrum in accordance with various embodiments of the present disclosure.

FIG. 6 is a flowchart that provides one example of a method for electric brain stimulator in a brain stimulator device in accordance with various embodiments of the present disclosure.

FIG. 7 illustrates an electric brain stimulator procedure in accordance with various embodiments of the present disclosure.

FIG. 8 illustrates the relationship between working memory value (K value) and electrical current in accordance with various embodiments of the present disclosure.

FIG. 9 illustrates another electric brain stimulator procedure in accordance with various embodiments of the present disclosure.

FIG. 10 illustrates the relationship between working memory value (K value) and electrical current in accordance with various embodiments of the present disclosure.

### DETAILED DESCRIPTION OF THE INVENTION

Having summarized various aspects of the present disclosure, reference will now be made in detail to the description of the disclosure as illustrated in the drawings. While the disclosure will be described in connection with these drawings, there is no intent to limit it to the embodiment or embodiments disclosed herein. On the contrary, the intent is to cover all alternatives, modifications and equivalents included within the spirit and scope of the disclosure as defined by the appended claims.

The present invention discloses a method implemented in a brain stimulator device for electric brain stimulator. It is understood that the method provides merely an example of the many different types of functional arraignments that may be employed to implement the operation of the various components of a system for electric brain stimulator, a computer system, a multiprocessor computing device, and so forth. The execution steps of the present invention may include application specific software which may store in any portion or component of the memory including, for example, random access memory (RAM), read-only memory (ROM), hard drive, solid-state drive, magneto optical (MO), IC chip, USB flash drive, memory card, optical disc, or other memory components.

For some embodiments, the system comprises a display device, a processing unit, a memory, an input device and a storage medium. The input device used to provide data such as image, text or control signals to an information processing system such as a computer or other information appliance. In accordance with some embodiments, the storage medium such as, by way of example and without limitation, a hard drive, an optical device or a remote database server coupled to a network, and stores software programs. The memory typically is the process in which information is encoded, stored, and retrieved etc. The processing unit performs data calculations, data comparisons, and data copying. The display device is an output device that visually conveys text, graphics, and the brain amplitude modulation spectrum. Information shown on the display device is called soft copy because the information exists electronically and is displayed for a temporary period of time. The display device includes CRT monitors, LCD monitors and displays, gas plasma monitors, and televisions. In accordance with such embodiments of present invention, the software programs are stored in the memory and executed by the processing unit when the computer system executes the method for electric brain stimulator. Finally, information provided by the processing unit, and presented on the display device or stored in the storage medium.

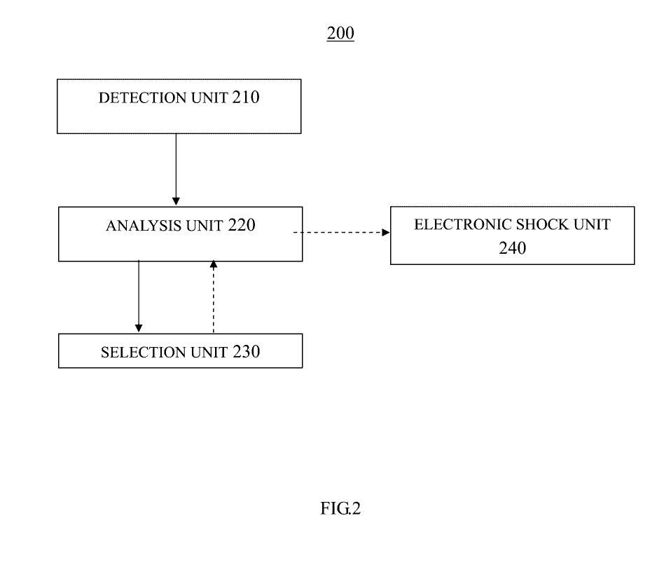

FIG. 2 is a block diagram of a system in which embodiments of electric brain stimulator for outputting a current via brainwave data analysis may be implemented in accordance with various embodiments of the present disclosure. The system of electric brain stimulator 200 comprises a detection unit 210, an analysis unit 220, a selection unit 230 and an electronic shock unit 240.

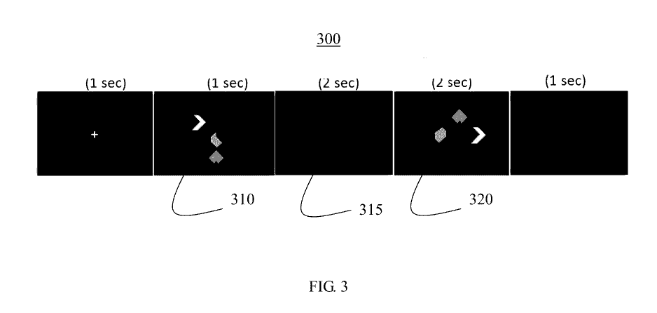

In one embodiment, FIG. 3 illustrates the example the Binding Visual Working

Memory Paradigm 300 in accordance with various embodiments of the present disclosure. In this task, participants are requested to see a study array 310 (usually 1000-2000 ms) first, then see a test array 320 (usually 1000-2000 ms) after a short retention interval 315, for example, 2 seconds, and further participants are requested to indicate any changes between study array 310 and test array 320. Participants performed the relationship between color-shape binding change detection tasks in Color-Shape Binding Visual Working Memory assignment.

The detection unit 210 is for acquiring a plurality of brainwave data. Participants memorize the study array 310 first, after a short retention interval 315, then participants are required to memorize the test array 320 while their brainwave data are recorded. The plurality of brainwave data is electroencephalography (EEG) or magnetoencephalography (MEG) recorded from multiple electrodes placed on the scalp.

The analysis unit 220 is connected to the detection unit 210 for analyzing the plurality of brainwave data to obtain a brain functional amplitude modulation spectrum. The brain functional amplitude modulation spectrum provides a power relation value or a correlation value of behavior and cognitive function for a frequency range and an amplitude-frequency range on different brain sites. The analysis unit 220 outputs an alternating current signal, and wherein the alternating current signal is generated based on a first cosine function of the first alternating current frequency, a second cosine function of the second alternating current frequency, or a combination of the first cosine function and the second cosine function.

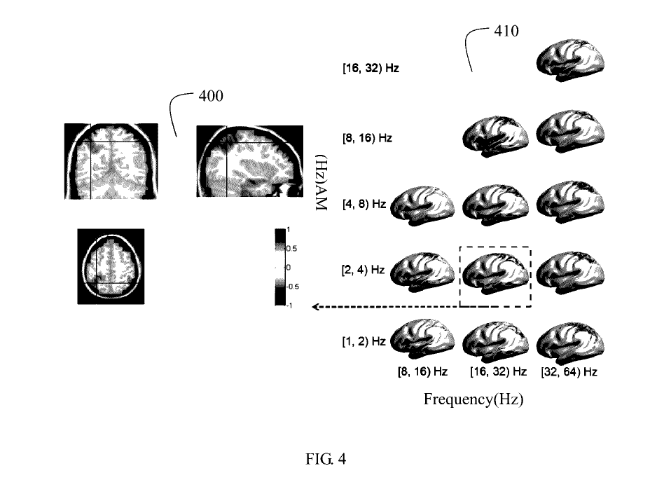

FIG. 4 illustrates a brain functional amplitude modulation spectrum 410 with correlation between power and K-value in accordance with various embodiments of the present disclosure. The brain functional amplitude modulation spectrum 410 provides tomographies, for example, dynamic EEG-based projected brain tomography Imager (deepBTGI) for six high performers and six low performers give a clear indication for the determination and optimization of transcranial alternating current stimulation (tACS) parameters, montages, and modulation depth and patterns. The brain functional amplitude modulation spectrum 410 provides tomographies between the frequency range from 8 to 64 Hz and the amplitude-frequency range from 1 to 32 Hz. The K-value is a working memory ability index. The different shades of colors in the tomography present different correction coefficients and a result of statistical analysis (p<0.01 cluster-based permutation (right-tailed)). With further reference to FIG. 4, an orthographic view 400 provides a dyadic tomography for further diagnosis of brain regions. The orthographic view 400 is a tomography based on the amplitude-frequency range from 2 to 4 Hz corresponding to the frequency range from 16 to 32 Hz.

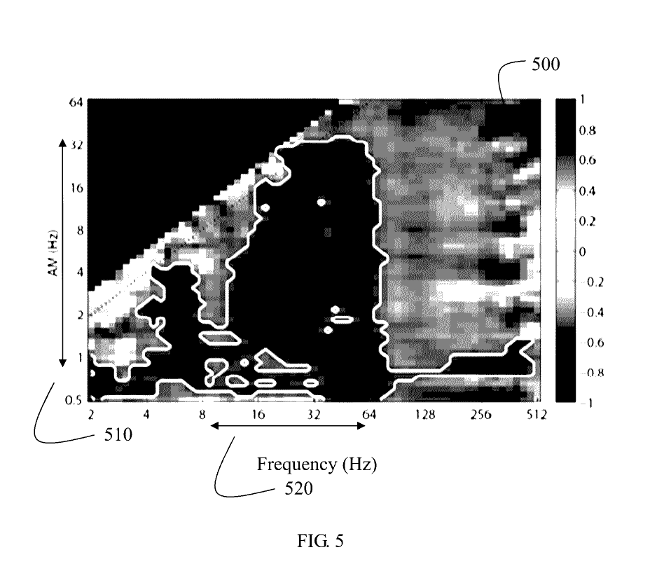

FIG. 5 illustrates another brain functional amplitude modulation spectrum in accordance with various embodiments of the present disclosure. The analysis unit 220 analyzes the correlation between HHS power and K value score of working memory in the brain functional amplitude modulation spectrum 500.

In one embodiment, take the power relation value of left posterior parietal cortex (LPPC). The analysis unit 220 analyzes the working memory of the participant, wherein the areas circled by white contours is a significant correlation (p<0.05 two-tailed) obtained by a Cluster-Based Nonparametric Permutation test.

The brain functional amplitude modulation spectrum 500 provides a mean value of hit of holo-hilbert spectral (HHS) power for memory retention during the retention interval in left posterior parietal cortex which shows the changes in the brain of the participant after see the study array. The analysis unit 220 analyzes a correlation analysis between HHS power and K-value in the brain functional amplitude modulation spectrum 500, wherein the K value is behavioral index of working memory capacity.

The analysis unit 220 outputs the alternating current signal based on the power relation value between the frequency range and the amplitude-frequency range on different brain sites in the brain amplitude modulation spectrum 500. The brain functional amplitude modulation spectrum 500 comprises a first alternating current frequency range 510 and a second alternating current frequency range 520, wherein the first alternating current frequency range 510 is the amplitude-frequency range in the brain functional amplitude modulation spectrum 500, for example, from 0.5 Hz to 32 Hz, and the second alternating current frequency range 520 is the frequency range in the brain functional amplitude modulation spectrum 500, for example, from 8 H to 64 Hz. Furthermore, the alternating current signal is generated based on a first cosine function of the first alternating current frequency, a second cosine function of the second alternating current frequency, or a linear or nonlinear combination of the first cosine function and the second cosine function.

In one embodiment, the alternating current signal based on the second cosine function of the second alternating current frequency is calculated by the analysis unit 220 according to the following expression:

f(t)=I₀+cos(f₂*2πt)

wherein I₀ is direct current and f2 is the second alternating current frequency.

In one embodiment, the alternating current signal based on the product of the first cosine function of the first alternating current frequency and the second cosine function of the second alternating current frequency is calculated by the analysis unit 220 according to the following expression:

f(t)=I₀+cos(f₁*πt)cos(f₂*2πt)

wherein J₀ is direct current, f1 is the first alternating current frequency and f2 is the second alternating current frequency.

In one embodiment, the alternating current signal based on the first cosine function of the first alternating current frequency is calculated by the analysis unit 220 according to the following expression:

f(t)=I₀+cos(f₁*2πt) or f(t)=I₀+cos(f₁*πt)

wherein J₀ is direct current and f1 is the first alternating current frequency.

In one embodiment, the alternating current signal based on the product of the first cosine function of the first alternating current frequency and the second cosine function of the second alternating current frequency is calculated by the analysis unit 220 according to the following expression:

f(t)=[I₀+cos(f₁*πt)]*cos(f₂*2πt) or f(t)=[I₀+cos(f₁*2πt)]*cos(f₂*2πt)

wherein J₀ is direct current, f1 is the first alternating current frequency and f2 is the second alternating current frequency.

The selection unit 230 is connected to the analysis unit 220 for determining a first alternating current frequency based on the amplitude-frequency range corresponding to a maximum power relation value or a maximum correlation value of behavior and cognitive function in the brain functional amplitude modulation spectrum. The selection unit 230 determines a second alternating current frequency based on the frequency range corresponding to a maximum power relation value or a maximum correlation value of behavior and cognitive function in the brain functional amplitude modulation spectrum. An electronic shock unit 240 is connected to the analysis unit 220 for outputting an electrical current directly applied the scalp, wherein the electrical current is corresponding to the alternating current signal.

The maximum power of relation value or a maximum correlation value of behavior and cognitive function is a range of values (interval) not a fixed value in the brain functional amplitude modulation spectrum. Therefore, the first alternating current frequency and the second alternating current frequency is dynamic change in the range of values.

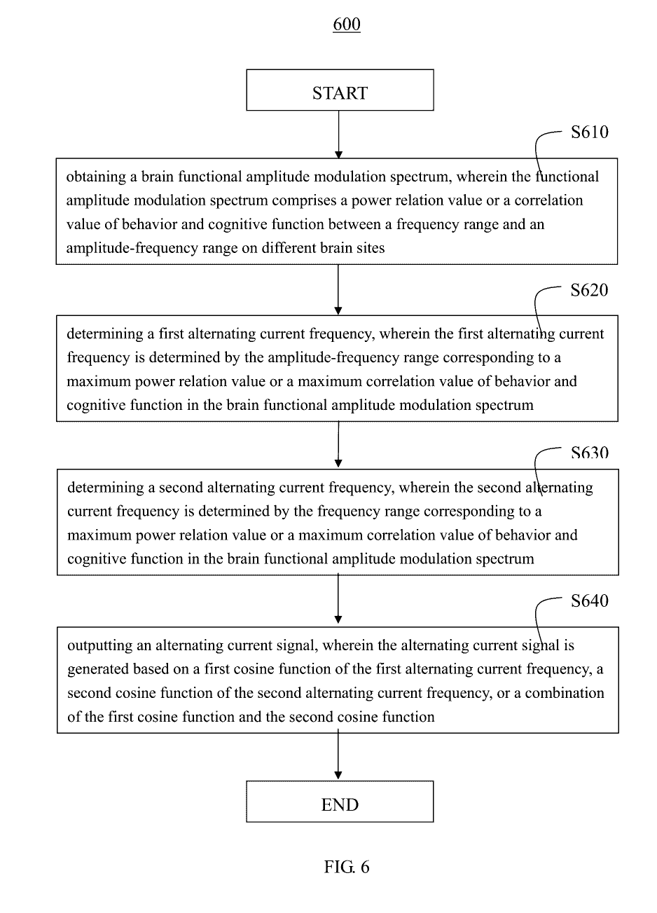

In FIG. 6 is a flowchart that provides one example of a method 600 for electric brain stimulator in a brain stimulator device, according to some embodiments. First of all, in step S610, the analysis unit 220 obtains a brain functional amplitude modulation spectrum, comprises steps below. Although the flowchart of FIG. 6 shows a specific order of execution, it is understood that the order of execution may differ from that which is depicted. For example, the order of execution of two or more blocks may be scrambled relative to the order shown. Also, two or more blocks shown in succession in FIG. 6 may be executed concurrently or with partial concurrence. It is understood that all such variations are within the scope of the present disclosure. The detection unit 210 receives a plurality of brainwave data, wherein the plurality of brainwave data is collected from a plurality of brain sites of a participant.

Then, the analysis unit 220 decomposes one of brainwave data, wherein the plurality of brainwave data is electroencephalography or magnetoencephalography recorded from multiple electrodes placed on the scalp. The analysis unit 220 selects one of brainwave data to obtain a plurality of intrinsic mode functions based on performing a mode decomposition method, wherein the plurality of intrinsic mode functions are an amplitude value changes over time of the brainwave data in each different frequency scale. The analysis unit 220 selects another of the brainwave data, executes the last step repeatedly until obtaining the plurality of intrinsic mode functions from all of the brainwave data. The plurality of intrinsic mode functions is classified in the same frequency scale into a plurality of frequency ranges corresponding to the different brain sites.

A source reconstruction method is performed to transform the plurality of intrinsic mode functions in the same frequency scale into a source space to obtain a plurality of source intrinsic mode functions (source IMFs) corresponding to the different brain sites. Then, selecting another one of the source intrinsic mode functions and executes the last step repeatedly until obtaining the plurality of source intrinsic mode functions from all of the source intrinsic mode functions. One of the source intrinsic mode functions is selected and takes an absolute value of the source intrinsic mode function to produce an amplitude envelope line comprising all maxima of the absolute value.

Further, the mode decomposition method is performed to obtain the plurality of source first-layer amplitude intrinsic mode functions of the amplitude envelope line. Another one of the source intrinsic mode functions and executes the last step repeatedly, until obtaining the plurality of source first-layer amplitude intrinsic mode functions from all of the source intrinsic mode functions, wherein the plurality of source first-layer amplitude intrinsic mode functions are a value changes over time of the amplitude envelope line in each different amplitude-frequency scale. The plurality of source first-layer amplitude intrinsic mode functions is classified in the same amplitude frequency scale into a plurality of amplitude-frequency ranges corresponding to the different brain sites.

A source reconstruction method, for example, beamformer, minimum norm estimation (MNE), eLORETA or multiple sparse priors is performed and utilizing a forward model, for example, spherical model, boundary element model, and finite element model on sources over a 2D cortical mesh, 3D cortical mesh or a 3D grid derived from a template (e.g. MNI template) or a 3D structure magnetic resonance imaging (MRI) to transform the plurality of intrinsic mode functions in the same frequency scale into a source space to obtain a plurality of source intrinsic mode functions corresponding to the different brain sites.

Then, another one of the source intrinsic mode functions is selected and executes the last step repeatedly, until obtaining the plurality of source first-layer amplitude intrinsic mode functions from all of the source intrinsic mode functions. The brain functional amplitude modulation spectrum provides power relation values between the frequency range and the amplitude-frequency range on different brain sites.

In an embodiment, the mode decomposition method may include by way of example and without limitation, such as empirical mode decomposition (EMD), ensemble empirical mode decomposition (EEMD) and conjugate adaptive dyadic masking empirical mode decomposition (CADM-EMD). The mode decomposition method decomposes the brainwave data to obtain the plurality of intrinsic mode functions. Beside the mode decomposition method mentions above, the plurality of intrinsic mode functions may include by way of example and without limitation, decomposed by adaptive filtering or optimal basis pursue.

In step S620, the selection unit 230 selects a first alternating current frequency, wherein the selection unit 230 selects the first alternating current frequency based on correlation between the amplitude-frequency range and a maximum power relation value in the brain functional amplitude modulation spectrum.

In step S630, the selection unit 230 selects a second alternating current frequency, wherein the selection unit 230 selects the second alternating current frequency based on correlation between the frequency range and a maximum power of relation value in the brain functional amplitude modulation spectrum.

In step S640, the electronic shock unit 240 outputs an electrical current directly on to the scalp, wherein the electrical current corresponding to the alternating current signal, wherein the alternating current signal is generated based on a first cosine function of the first alternating current frequency, a second cosine function of the second alternating current frequency, or a linear or nonlinear combination of the first cosine function and the second cosine function.

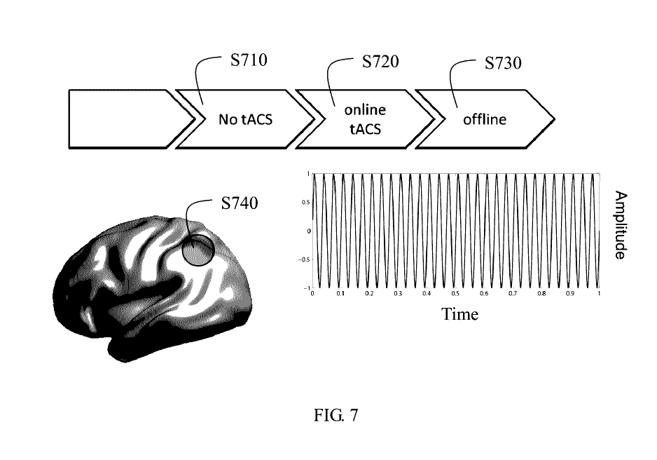

Please refer FIG. 7 which illustrates an electric brain stimulator procedure according to alternative embodiments of the present disclosure. A participate has an electrical current directly applied the scalp before the participate see the test array or while the participate see the test array, wherein the electrical current is 30 Hz based on the cosine function of the second alternating current frequency, for example, cos(2π*30t) according to the invasive brain stimulation techniques, for example, transcranial alternating current stimulation (tACS). The plurality of brainwave data is brainwave signal collected from multiple electrodes placed on the left posterior parietal cortex 740 of the participant. The plurality of brainwave signals are collected from three stage comprising No tACS 710, online tACS 720 and offline 730. In one embodiment, the brainwave signal can be transmitted wirelessly via a smart phone to a cloud based server for analysis and stimulus optimization.

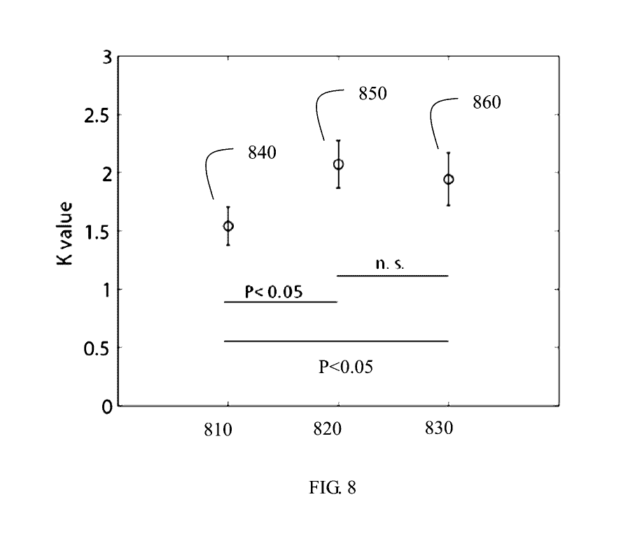

FIG. 8 illustrates the relationship between working memory value (K value) and electrical current according to alternative embodiments of the present disclosure. FIG. 8 includes Pre (not sending an electric current into the brain) 810, tACS (sending an electric current into the brain while seeing the test array) 820, Post (sending an electric current into the brain before seeing the test array) 830 and error bars 840, 850, 860 are standard error. Furthermore, n.s. is not significant and P<0.05 is significant. The Bonferroni Correction sets the significance difference between Post 830 and Pre 810. The invention provides holo-hilbert spectral analysis (HHSA) and the brain functional amplitude modulation spectrum for electric brain stimulator to improve memory ability. The fact that the participant's performance is indeed improved during the stimulating session, but that the effects wane immediately after the stimulation stops.

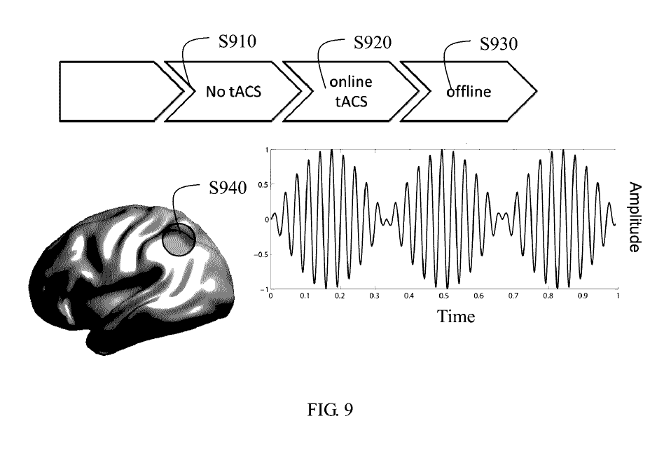

Please refer FIG. 9, illustrates another electric brain stimulator procedure according to alternative embodiments of the present disclosure. A participate has an electrical current directly applied the scalp before the participate see the test array or while the participate see the test array, wherein the alternating electrical current is 30 Hz and 3 Hz amplitude based on the product of the cosine function of the first alternating current frequency and the cosine function of the second alternating current frequency, for example, cos(3*πt) cos(30*2πt) according to the invasive brain stimulation techniques, for example, transcranial modulated alternative current stimulation (tMACS). The plurality of brainwave data is brainwave signal collected from multiple electrodes placed on the left posterior parietal cortex 940 of the participant. The plurality of brainwave signals are collected from three stage comprising No tMACS 910, online tMACS 920 and offline 930. In one embodiment, the brainwave signal can be transmitted wirelessly via a smart phone to a cloud based sever for analysis and stimulus optimization.

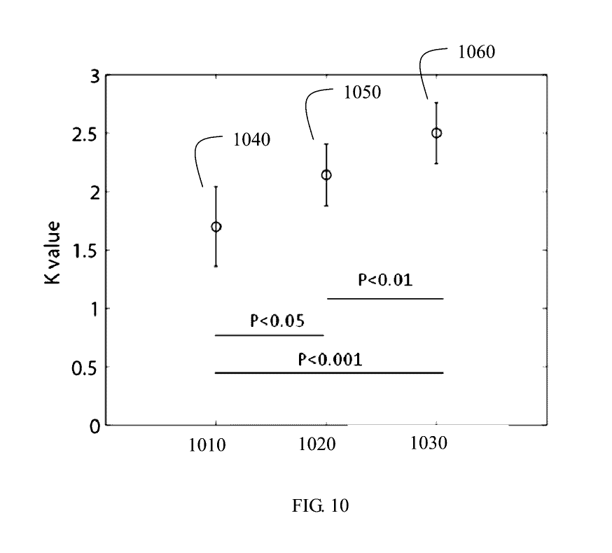

FIG. 10 illustrates the relationship between working memory value (K value) and electrical current according to alternative embodiments of the present disclosure. FIG. 10 includes Pre (not sending an electric current into the brain) 1010, tACS (sending an electric current into the brain while seeing the test array) 1020, Post (sending an electric current into the brain before seeing the test array) 1030 and error bars 1040, 1050, 1060 are standard error. The significance difference is based on comparison of P<0.05, P<0.01 and P<0.001. The invention provides holo-hilbert spectral analysis and the brain functional amplitude modulation spectrum for electric brain stimulator to improve the performance of these participants statistical significantly in the cognitive ability, for example, visual short-term memory (VSTM), measured by K value. And the effects are clearly not only during the stimulating session, but also retained long after the stimulating session.

The method and system for electric brain stimulator provides the parameters such as the montage, the stimulating wave amplitude, frequency and depth of modulation pattern for the hitMACS are determined objectively and quantitatively based in holo-hilbert spectral analysis and the brain functional amplitude modulation spectrum. The invention provides a method and system for diagnosis patients with the memory loss and memory impairment based on working memory value and outputs the alternating current signal which is a cosine function or a linear or nonlinear combination of cosine functions based on relation values in the brain functional amplitude modulation spectrum.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is :

1. A method for electric brain stimulator in a brain stimulator device, comprising:
(A) obtaining a brain functional amplitude modulation spectrum, wherein the brain functional amplitude modulation spectrum comprises a power relation value or a correlation value of behavior and cognitive function between a frequency range and an amplitude-frequency range on different brain sites;
(B) determining a first alternating current frequency, wherein the first alternating current frequency is determined by the amplitude-frequency range corresponding to a maximum power relation value or a maximum correlation value of behavior and cognitive function in the brain functional amplitude modulation spectrum;
(C) determining a second alternating current frequency, wherein the second alternating current frequency is determined by the frequency range corresponding to a maximum power relation value or a maximum correlation value of behavior and cognitive function in the brain functional amplitude modulation spectrum; and
(D) outputting an alternating current signal, wherein the alternating current signal is generated based on a first cosine function of the first alternating current frequency, a second cosine function of the second alternating current frequency, or a combination of the first cosine function and the second cosine function.

2. The method of claim 1, wherein the cosine functions of the alternating current signal are calculated according to the following expression:
f(t)=I₀+cos(f₁*πt)*cos(f₂*2πt)
wherein J₀ is direct current, f1 is the first alternating current frequency and f2 is the second alternating current frequency.

3. The method of claim 1, wherein the cosine functions of the alternating current signal are calculated according to the following expression:
f(t)=I₀+cos(f₂*2πt)
wherein J₀ is direct current and f2 is the second alternating current frequency.

4. The method of claim 1, wherein the cosine functions of the alternating current signal are calculated according to the following expression:
f(t)=I₀+cos(f₁*2πt) or f(t)=I₀+cos(f₁*πt)
wherein J₀ is direct current and f1 is the first alternating current frequency.

5. The method of claim 1, wherein the cosine functions of the alternating current signal are calculated according to the following expression:
f(t)=[I₂+cos(f₁*πt)]*cos(f₂*2πt) or f(t)=[I₀+cos(f₁*2πt)]*cos(f₂*2πt)
wherein J₀ is direct current, f1 is the first alternating current frequency and f2 is the second alternating current frequency.

6. The method of claim 1, wherein obtaining the brain functional amplitude modulation spectrum comprises:
(A1) obtaining a plurality of brainwave data, wherein the plurality of brainwave data is collected from a plurality of brain sites;
(A2) performing a mode decomposition method on one of the plurality of brainwave data, generating a plurality of intrinsic mode functions, wherein the plurality of intrinsic mode functions are an amplitude value changes over time of the brainwave data in each different frequency scale;
(A3) selecting another one of the brainwave data, repeating step (A2) until obtaining the plurality of intrinsic mode functions from all of the brainwave data;
(A4) classifying the plurality of intrinsic mode functions in the same frequency scale into a plurality of frequency ranges corresponding to the different brain sites;
(A5) based on a source reconstruction method to transform the plurality of intrinsic mode functions in the same frequency scale into a source space, obtaining a plurality of source intrinsic mode functions corresponding to the different brain sites;
(A6) selecting one of the source intrinsic mode functions, taking an absolute value of the source intrinsic mode function, then producing an amplitude envelope line comprising all maxima of the absolute value, and obtaining a plurality of source first-layer amplitude intrinsic mode functions of the amplitude envelope line based on performing the mode decomposition method, wherein the plurality of source first-layer amplitude intrinsic mode functions are a value changes over time of the amplitude envelope line in each different amplitude frequency scale;
(A7) selecting another one of the source intrinsic mode functions, repeating step (A6) until obtaining the plurality of source first-layer amplitude intrinsic mode functions from all of the source intrinsic mode functions;
(A8) classifying the plurality of source first-layer amplitude intrinsic mode functions in the same amplitude frequency scale into a plurality of amplitude frequency ranges corresponding to the different brain sites; and
(A9) generating the brain functional amplitude modulation spectrum based on the plurality of frequency ranges corresponding to the plurality of amplitude frequency ranges at same time.

7. The method of claim 6, wherein the plurality of brainwave data is electroencephalography (EEG) or magnetoencephalography (MEG) recorded from multiple electrodes placed on the scalp.

8. The method of claim 6, wherein the mode decomposition method comprises empirical mode decomposition, ensemble empirical mode decomposition or conjugate adaptive dyadic masking empirical mode decomposition.

9. The method of claim 6, wherein the source reconstruction method comprises beam former, minimum norm estimation, eLORETA or multiple sparse priors.

10. The method of claim 6, wherein the source space is a 2D cortical mesh or a 3D cortical mesh obtained by a spherical model, a boundary element model or a finite element model.

11. The method of claim 6, wherein the source space is a template or a 3D structure magnetic resonance imaging (MRI).

12. A system of electric brain stimulator, comprises:
a detection unit for acquiring a plurality of brainwave data;
an analysis unit connected to the detection unit for analyzing the plurality of brainwave data to obtain a brain functional amplitude modulation spectrum, wherein the brain functional amplitude modulation spectrum comprises a power relation value or a correlation value of behavior and cognitive function between a frequency range and an amplitude-frequency range on different brain sites and outputs an alternating current signal, wherein the alternating current signal is generated based on a first cosine function of the first alternating current frequency, a second cosine function of the second alternating current frequency, or a combination of the first cosine function and the second cosine function;
a selection unit connected to the analysis unit for determining a first alternating current frequency based on the amplitude-frequency range corresponding to a maximum power relation value or a maximum correlation value of behavior and cognitive function in the brain functional amplitude modulation spectrum, and determining a second alternating current frequency based on the frequency range corresponding to a maximum power of relation value or a maximum correlation value of behavior and cognitive function in the brain functional amplitude modulation spectrum; and
an electronic shock unit connected to the analysis unit for outputting a electrical current corresponding to the alternating current signal.

13. The system of claim 12, wherein the analysis unit calculates the cosine functions of the alternating current signal according to the following expression:
f(t)=I₀+cos(f₁*πt)*cos(f₂*2πt)
wherein J₀ is direct current, f1 is the first alternating current frequency and f2 is the second alternating current frequency.

14. The system of claim 12, wherein the analysis unit calculates the cosine functions of the alternating current signal according to the following expression:
f(t)=I₀+cos(f₂*2πt)
wherein J₀ is direct current and f2 is the second alternating current frequency.

15. The system of claim 12, wherein the analysis unit calculates the cosine functions of the alternating current signal according to the following expression:
f(t)=I₀+cos(f₁*2πt) or f(t)=I₀+cos(f₁*πt)
wherein J₀ is direct current and f1 is the first alternating current frequency.

16. The system of claim 12, wherein the analysis unit calculates the cosine function of the alternating current signal according to the following expression:
f(t)=[I₀+cos(f₁*πt)]*cos(f₂*2πt) or f(t)=[I₀+cos(f₁*2πt)]*cos(f₂*2πt)
wherein J₀ is direct current, f1 is the first alternating current frequency and f2 is the second alternating current frequency.
